(19) United States
(12) Patent Application Publication
(10) Pub. No.: US 2023/0101092 A1
(43) Pub. Date: Mar. 30, 2023

(54) RADIO FREQUENCY GENERATING DEVICE

(71) Applicant: TOKYO KEIKI INC., Tokyo (JP)

(72) Inventor: Koichi Murai, Tokyo (JP)

(21) Appl. No.: 17/910,027

(22) PCT Filed: Jan. 13, 2021
(86) PCT No.: PCT/JP2021/000780

§ 371 Date: Sep. 8, 2022

(30) Foreign Application Priority Data
Apr. 9, 2020 (JP) 2020-070185

(51) Int. Cl. H05H 1/46

(52) U.S. Cl. CPC H05H 1/4645; A61L 2/14

(57) ABSTRACT

An RF generating device 2, which attenuates harmonics in RF power amplified by an RF amplifier and supplied to plasma generating electrodes, includes an RF generation unit 201 that generates a composite wave by combining a fundamental wave with a compensation wave which is made by inverting a phase of a harmonic generated by inputting the fundamental wave.

### TECHNICAL FIELD

The embodiments of the present invention relate to a technique for attenuating harmonics related to generation of plasma.

### BACKGROUND ART

Plasma generating apparatuses that generate plasma by supplying radio frequency (RF) power to plasma generating electrodes are known. As the RF power supplied to a load that generates plasma (plasma load) is amplified by an amplifier, harmonics are generated by the nonlinearity of the amplifier. This necessitates the plasma generating apparatuses to be equipped, between the plasma load and the amplifier, with filters to attenuate harmonics.

In recent years, a method of switching the frequency of RF power supplied to the plasma load with plural frequencies is used in plasma generating apparatuses, and the most common implementation is to combine two RF power sources having frequencies of 12.88-14.24 MHz (fundamental wave 1) and 38.65-42.71 MHz (fundamental wave 2), respectively. In such cases, as the frequency of the third harmonic of the fundamental wave 1 is close to the frequency of the fundamental wave 2 and may cause malfunctions, the third harmonic needs to be attenuated by a filter.

In the related art, a microwave power supply for plasma generation is known that outputs microwaves while periodically switching the path of the microwave output from the microwave power supply to multiple antennas using a microwave switch (see Patent Document 1).

### CITATION LIST

### Patent Document

Patent document 1: JPH9-274999

### DISCLOSURE OF THE INVENTION

### Problems to be Solved by the Invention

In plasma generating apparatuses that use plural frequencies of RF power, as described above, the same RF amplifier can be used to amplify different frequencies of RF power, but it is difficult to electrically switch the passband of the filter because of the need to handle high power in the range of kilowatts.

Therefore, even if an RF amplifier can handle plural frequencies, a pair of RF amplifier and filter must be provided for each frequency resulting in increasing cost of switching frequencies in plasma generating apparatuses.

The purpose of the present invention is to solve the aforementioned problems and to provide a technique for attenuating harmonics in a plasma generating apparatus at a lower cost.

### Means for Solving the Problems

In order to solve the above-mentioned problems, the RF generating device of this embodiment is an RF generating device that attenuates harmonics in RF power amplified by an RF amplifier and supplied to plasma generating electrodes, and includes an RF generation unit that generates a composite wave by combining a fundamental wave with a compensation wave which is made by inverting a phase of a harmonic generated by inputting the fundamental wave.

### Advantageous Effects of the Invention

The present invention allows a plasma generating apparatus to attenuate harmonics at a lower cost.

### BRIEF DESCRIPTION OF THE DRAWINGS

FIG. 1 is a schematic block diagram showing the configuration of the plasma generating apparatus.

FIG. 2 is a schematic block diagram showing the hardware configuration of the RF generating device.

FIG. 3 is a block diagram showing the functional configuration of the RF generating device.

FIG. 4 is a flowchart showing the operation of the RF generating device.

FIG. 5 shows the input and output waveforms in a distorted state.

FIG. 6 shows the input and output waveforms in a state of improved distortion.

### BEST MODE FOR CARRYING OUT THE INVENTION

The following is a description of the embodiment of the invention with reference to the drawings.

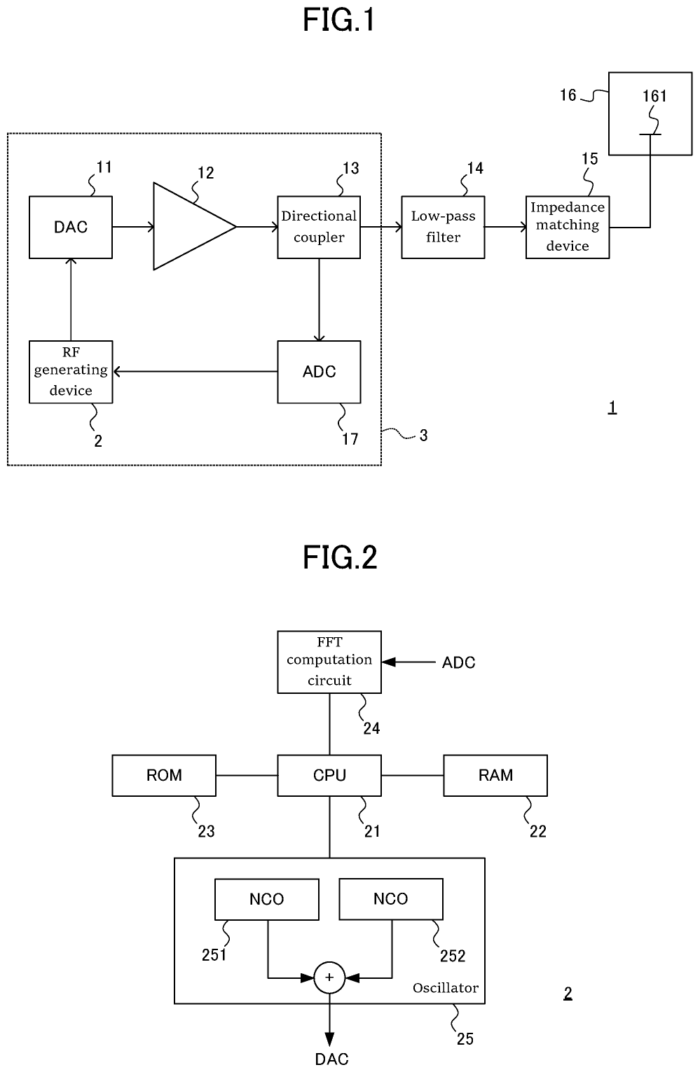

(Configuration of the plasma generating apparatus) The configuration of the plasma generating apparatus for this embodiment will be described. FIG. 1 is a schematic block diagram showing the configuration of the plasma generating apparatus of this embodiment.

As shown in FIG. 1, the plasma generating apparatus 1 in this embodiment includes an RF generating device 2, a DAC (Digital to Analog Converter) 11, an RF amplifier 12, a directional coupler 13, a low-pass filter 14, an impedance matching device 15, a plasma space 16, and an ADC (Analog to Digital Converter 17. Among these, the RF generating device 2, DAC 11, RF amplifier 12, directional coupler 13, and ADC 17 are configured as part of the RF power supply 3 that outputs RF power to generate plasma.

The RF generating device 2 generates two fundamental waves with different frequencies that are used to generate RF power. The RF generating device 2 generates a fundamental wave having the lower frequency (the first fundamental wave) of the two fundamental waves and a compensation wave that is made by inverting the phase of a harmonic of the first fundamental wave having a frequency closest to that of the other fundamental wave (the second fundamental wave), and generates a composite wave by combining the compensation wave and the first fundamental wave.

The RF generating device 2 attenuates the harmonics of the first fundamental wave. The frequency of the second fundamental wave is approximately three times the frequency of the first fundamental wave, and the RF generating device 2 generates a compensation wave that is made by inverting the phase of the third harmonic of the first fundamental wave.

The DAC 11 outputs an RF signal to the RF amplifier 12, which is an analog signal D/A-converted from the digital data of the first and second fundamental waves, respectively, generated by the RF generating device 2. The RF amplifier 12 outputs the RF power obtained by amplifying the RF signal output from the DAC 11 to the directional coupler 13.

Directional coupler 13 outputs the RF power output from the RF amplifier 12 to the low-pass filter 14, while the same RF power is also branched and output to the ADC 17. The low-pass filter 14 passes RF power in the frequency range that includes the frequencies of the first and second fundamental waves, while attenuating harmonics generated in the RF power of the second fundamental wave, which has a higher frequency than that of the first fundamental wave.

The impedance matching device 15 matches the impedance of the plasma load generated in the plasma space 16 to the impedance of the transmission line on the RF power supply 3 side. The plasma space 16 has a space for generating plasma inside, and plasma generating electrodes 161 that generate plasma in the plasma space 16 when RF power is supplied are provided within the plasma space 16. The ADC 17 outputs A/D-converted digital data of the RF power output from the directional coupler 13 to the RF generating device 2.

(Configuration of the RF generating device)

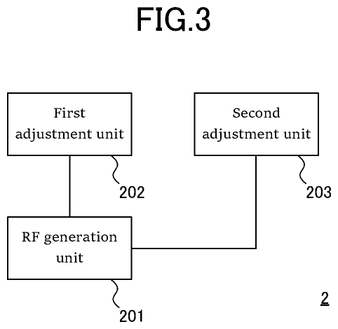

The hardware and functional configurations of the RF generating device are described below. FIGS. 2 and 3 are block diagrams showing the hardware and functional configurations of the RF generating device, respectively.

As shown in FIG. 2, the RF generating device 2's hardware includes a CPU

(Central Processing Unit) 21, a RAM (Random access memory) 22, a ROM (Read Only Memory) 23, an FFT (Fast Fourier Transform) computation circuit 24 and an oscillator 25. In this embodiment, the RF generating device 2 is configured in an FPGA (Field Programmable Gate Array) having a CPU 21, a RAM 22, and a ROM 23.

The CPU 21 and RAM 22 work together to perform various functions described below, and the ROM 23 stores various data used in the processes performed in the various functions. The FFT computation circuit 24 computes the amplitudes and phases of each of the first fundamental wave and the third harmonic of the first fundamental wave by Fourier-transforming the RF power A/D-converted by the ADC 17.

The oscillator 25 has an NCO (Numerically controlled oscillator) 251 that outputs the first fundamental wave and an NCO 252 that outputs the compensation wave described below, and outputs a composite wave, which is digital waveform signals made by combining these two NCO outputs, to DAC 11.

As shown in FIG. 3, the RF generating device 2 has functional units including an RF generation unit 201, a first adjustment unit 202, and a second adjustment unit 203. The RF generation unit 201 generate a composite wave by determining the amplitudes and phases of each of the first fundamental wave and the compensation wave based on the first fundamental wave information and the compensation wave information stored in the ROM 23, and outputting them to the oscillator 25. The first adjustment unit 202 adjusts the amplitude and phase of the compensation wave determined by the RF generation unit 201 based on the harmonic component data of the third harmonic of the fundamental wave computed by the FFT computation circuit 24. The second adjustment unit 203 adjusts the amplitudes of the fundamental wave and compensation waves based on the amplitude of the composite wave.

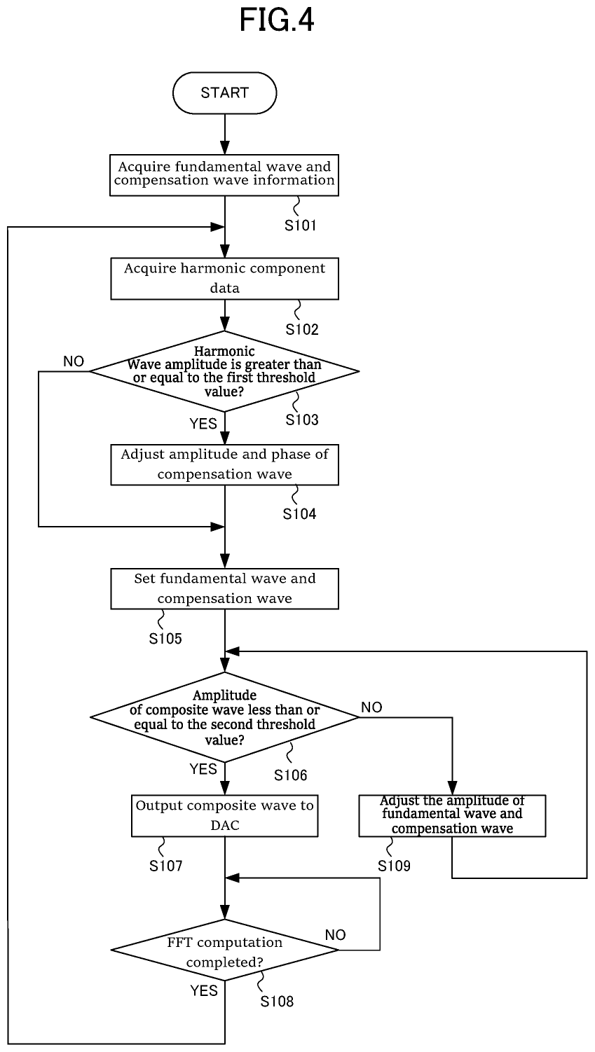

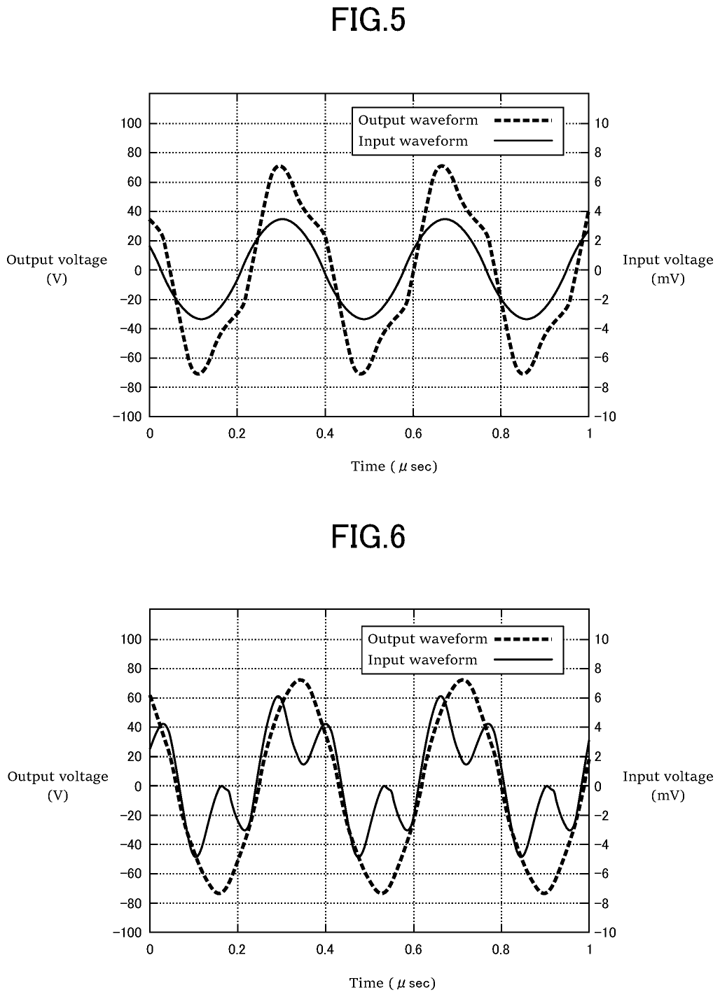

(Operation of the RF generating device) The operation of the RF generating device for generation of the first fundamental wave is described below. FIG. 4 is a flowchart showing the operation of the RF generating device. FIGS. 5 and 6 show input and output waveforms in a distorted state, and in a state of improved distortion, respectively. Note that the operations shown in FIG. 4 are performed every FFT computation that requires multiple samplings, e.g., 256 samplings.

As shown in FIG. 4, the RF generation unit 201 acquires the fundamental wave information and compensation wave information from the ROM 23 stored in advance (S101). Then, the compensation wave information will be explained. The compensation wave information is information indicating the amplitudes and phases of the harmonics obtained by measuring in advance the distortion characteristics of the RF amplifier 12 when an RF power of the first fundamental wave is input. In this embodiment, the compensation wave information indicates the amplitude and phase of the compensation wave which is made by inverting the phase of the third harmonic of the first fundamental wave as described above.

Next, the first adjustment unit 202 acquires a harmonic component data computed by the FFT computation circuit 24 (S102). The initial value of the harmonic component data, which is the value before the FFT computation is started, is set to 0.

Next, the first adjustment unit 202 determines whether the amplitude of the harmonic indicated by the harmonic component data is greater than or equal to a first threshold value (S103). The first threshold value should preferably be set to a value acceptable to the plasma generating apparatus 1, i.e., to the amplitude of harmonics that do not cause malfunctions in the plasma generating apparatus 1.

When the amplitude of the harmonics is greater than or equal to the first threshold value (S103, YES), the first adjustment unit 202 adjusts the amplitude and phase of the compensation wave so that the third harmonic in the RF power output from the RF amplifier 12 is attenuated (S104), and the RF generation unit 201 sets the amplitude and phase of the first fundamental wave to the NCO 251 and the amplitude and phase of the compensation wave to the NCO 252 (S105).

On the other hand, when the amplitude of the harmonics is not greater than nor equal to the first threshold value (S103, NO), the first adjustment unit 202 performs no adjustments to the compensation wave, and the RF generation unit 201 sets the fundamental wave and compensation waves to NCO 251 and NCO 252 (S105).

After combining the fundamental wave and the compensation wave, the second adjustment unit 203 determines whether the amplitude of the composite wave is less than or equal to the second threshold value (S106). In the above, the composite wave that the second adjustment unit 203 examines is the composite wave data computed based on the respective amplitudes and phases of the first fundamental wave and compensation wave set in NCO 251 and NCO 252, and the second threshold value should be set to the maximum amplitude at which the composite wave data does not saturate the DAC 11.

When the amplitude of the composite wave is less than or equal to the second threshold value (S106, YES), the RF generation unit 201 outputs the composite wave data to DAC 11 by having NCO 251 and NCO 252 output the first fundamental wave and the compensation wave (S107), and determines whether the FFT computation is completed (S108).

When the FFT computation is completed (S108, YES), the first adjustment unit 202 again acquires the harmonic component data computed by the FFT computation circuit 24 (S102); on the other hand, when the FFT computation is not completed (S108, NO), the RF generation unit 201 again determines whether the FFT computation is completed (S108).

When the amplitude of the composite wave is not less than nor equal to the second threshold value in the step S106 (S106, NO), the second adjustment unit 203 adjusts the amplitudes of the fundamental wave and compensation waves set in NCO 251 and NCO 252, respectively, so that the amplitude of the composite wave is attenuated to levels that do not saturate the DAC 11 (S109) and again determines whether the amplitude of the composite wave is less than or equal to the second threshold value (S106).

Thus, by applying a pre-distortion process on the RF signal at the input of the RF amplifier 12 based on the compensation wave defined by the predetermined compensation wave information and/or the third harmonic component in the RF power output from the RF amplifier 12, the distortion in the output waveform shown in FIG. 5 can be improved as shown in FIG. 6.

In this embodiment, the third harmonic is attenuated, but other harmonics may be attenuated. Only either one of the compensation wave or the harmonics in the RF power may be used to attenuate the harmonics. The plasma generating apparatus 1 of the embodiment described above having the RF generating device 2 may be used for a plasma sterilization device, a plasma light source, etc., for example.

The embodiments of the present invention have been presented by way of example only, and are not intended to limit the scope of the invention. The novel embodiments described herein may be embodied in a variety of other forms;

furthermore, various omissions, substitutions and changes may be made without departing from the spirit of the invention. The embodiments and modifications are included in the scope or spirit of the present invention and in the appended claims and their equivalents.

### REFERENCE SIGNS LIST

1 Plasma generating apparatus
2 RF generating device
201 RF generation unit
202 First adjustment unit
203 Second adjustment unit

1. A radio frequency (RF) generating device that attenuates harmonics in RF power amplified by an RF amplifier and supplied to plasma generating electrodes, comprising:
an RF generation unit that generates a composite wave by combining a fundamental wave with a compensation wave which is made by inverting a phase of a harmonic generated by inputting the fundamental wave.

2. The RF generating device according to claim 1, wherein the compensation wave is generated based on harmonic information, which is stored in a memory device in advance and related to characteristics of the RF amplifier.

3. The RF generating device according to claim 1, further comprising:
a first adjustment unit that adjusts an amplitude and a phase of the compensation wave so as to attenuate an amplitude of a harmonic wave when the amplitude of the harmonic wave in the RF power is greater than or equal to a predetermined first threshold value.

4. The RF generating device according to claim 3, further comprising:
a second adjustment unit that adjusts an amplitude of the fundamental wave and the amplitude of the compensation wave so that an amplitude of the composite wave is attenuated when the amplitude of the composite wave exceeds a second threshold value that is different from the predetermined first threshold value.

5. The RF generating device according to claim 1, wherein:
the plasma generating electrodes are selectively supplied with RF power of a first fundamental wave or a second fundamental wave whose frequency is higher than a frequency of the first fundamental wave,
the harmonic is a harmonic in the RF power of the first fundamental wave and is a harmonic whose frequency is closest to the frequency of the second fundamental wave, and
the RF generation unit generates the composite wave by combining the first fundamental wave with the compensation wave which is made by inverting a phase of the harmonic.

6. The RF generating device according to claim 2, further comprising:
a first adjustment unit that adjusts an amplitude and a phase of the compensation wave so as to attenuate an amplitude of a harmonic wave when the amplitude of the harmonic wave in the RF power is greater than or equal to a predetermined first threshold value.

7. The RF generating device according to claim 6, further comprising:
a second adjustment unit that adjusts an amplitude of the fundamental wave and the amplitude of the compensation wave so that an amplitude of the composite wave is attenuated when the amplitude of the composite wave exceeds a second threshold value that is different from the predetermined first threshold value.

8. The RF generating device according to claim 2, wherein:
the plasma generating electrodes are selectively supplied with RF power of a first fundamental wave or a second fundamental wave whose frequency is higher than a frequency of the first fundamental wave,
the harmonic is a harmonic in the RF power of the first fundamental wave and is a harmonic whose frequency is closest to the frequency of the second fundamental wave, and
the RF generation unit generates the composite wave by combining the first fundamental wave with the compensation wave which is made by inverting a phase of the harmonic.

9. The RF generating device according to claim 3, wherein:
the plasma generating electrodes are selectively supplied with RF power of a first fundamental wave or a second fundamental wave whose frequency is higher than a frequency of the first fundamental wave,
the harmonic is a harmonic in the RF power of the first fundamental wave and is a harmonic whose frequency is closest to the frequency of the second fundamental wave, and
the RF generation unit generates the composite wave by combining the first fundamental wave with the compensation wave which is made by inverting a phase of the harmonic.

10. The RF generating device according to claim 4, wherein:
the plasma generating electrodes are selectively supplied with RF power of a first fundamental wave or a second fundamental wave whose frequency is higher than a frequency of the first fundamental wave,
the harmonic is a harmonic in the RF power of the first fundamental wave and is a harmonic whose frequency is closest to the frequency of the second fundamental wave, and
the RF generation unit generates the composite wave by combining the first fundamental wave with the compensation wave which is made by inverting a phase of the harmonic.

11. The RF generating device according to claim 6, wherein:
the plasma generating electrodes are selectively supplied with RF power of a first fundamental wave or a second fundamental wave whose frequency is higher than a frequency of the first fundamental wave,
the harmonic is a harmonic in the RF power of the first fundamental wave and is a harmonic whose frequency is closest to the frequency of the second fundamental wave, and
the RF generation unit generates the composite wave by combining the first fundamental wave with the compensation wave which is made by inverting a phase of the harmonic.

12. The RF generating device according to claim 7, wherein:
the plasma generating electrodes are selectively supplied with RF power of a first fundamental wave or a second fundamental wave whose frequency is higher than a frequency of the first fundamental wave,
the harmonic is a harmonic in the RF power of the first fundamental wave and is a harmonic whose frequency is closest to the frequency of the second fundamental wave, and
the RF generation unit generates the composite wave by combining the first fundamental wave with the compensation wave which is made by inverting a phase of the harmonic.
